(19) United States
(12) Patent Application Publication
(10) Pub. No.: US 2008/0085527 A1
(43) Pub. Date: Apr. 10, 2008

(54) METHOD FOR MEASURING UREA

(75) Inventors: Hsiao-Chung Tsai, Changhua County (TW); Su-Jan Lee, Taipei City (TW); Tzu-I Wu, Changhua County (TW); Clifford Chenghwa Hou, Chesterfield, MO (US); Ian Liau, Taipei City (TW); Ke-Shieng Yang, Taipei City (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignees: INDUSTRIAL TECHNOLOGY RESEARCH INSTITUTE, Hsinchu (TW); APEX BIOTECHNOLOGY CORP., Hsinchu (TW)

(21) Appl. No.: 11/952,368

(22) Filed: Dec. 7, 2007

Related U.S. Application Data
(63) Continuation of application No. 11/175,099, filed on Jul. 5, 2005.

(30) Foreign Application Priority Data
Dec. 31, 2004 (TW) 93141719

(51) Int. Cl. C12Q 1/66

(52) U.S. Cl. 435/8

(57) ABSTRACT

Luminescence-based recipes for the measurement of creatinine, urea, uric acid, or glucose, and device using the same are provided.

### CROSS REFERENCE TO RELATED APPLICATIONS

This application is a continuation of pending U.S. patent application Ser. No. 11/175,099, filed Jul. 5, 2005 and entitled “Luminescence-based recipe and device using same”.

### BACKGROUND

The invention relates to a luminescence-based recipe and device using the same. More particularly, the invention relates to one-step, luminescence-based recipes for the measurement of creatinine, urea, uric acid, or glucose, and device using the same.

Biochemical analysis of small molecules is a routine procedure for health examination. Based on the analysis, physiological functions such as kidney, liver, or cardiovascular functions of a patient can be assessed by a physician. Present analysis is mainly based on absorbance or fluorescence which requires a specific light source and is not suitable for household or personal applications. Luminescence analysis is highly sensitive and relatively simple in design, and more particularly, most physiological markers or metabolites can be detected by luminescence analysis. Luminescence analysis can be, therefore, used in the development of fast analysis platform, or in the combination of optical sensors and micro-electro-mechanical system (MEMS) to design a portable physiological detector for personal health management.

The present luminescence-based physiological detector requires large amount of samples and can not be easily manipulated by non-professional persons. In addition, the difficulties of serum separation, matrix interference, sensitivity, reproducibility, and simplified machinery design are still problems to be solved. It is, therefore, still a need to develop one-step luminescence analysis and device for multiple analysts.

### SUMMARY

Accordingly, luminescence-based recipes for the measurement of creatinine, urea, uric acid, or glucose are provided.

An embodiment of the luminescence-based recipe for the measurement of creatinine comprises 0.01˜150 U/mL of creatininase, 0.01-150 U/mL of creatine kinase, 1×10⁻⁶˜5×10⁻² mg/mL of firefly luciferase, 0.1˜5000 μM of luciferin, 1 μM˜20 mM of MgSO₄, 0.1˜5000 μM of ATP, 0˜1% of BSA, 0˜50 mM of DTT (1,4-dithioerythritol)), and 5˜200 mM of buffer at pH6˜8.

An embodiment of the luminescence-based recipe for the measurement of urea comprises 0.01˜100 U/mL of urea amidolyase (URL), 1×10⁻⁶˜5×10⁻² mg/mL of firefly luciferase, 0.1˜5000 μM of luciferin, 1 μM˜20 mM of MgSO₄, 0.1˜5000 μM of ATP, 0˜100 mM of KCl, 0˜100 mM of NaHCO₃, 0˜20 mM of EGTA, 0˜1% of BSA, 0˜50 mM of DTT (1,4-dithioerythritol)), and 5˜200 mM of buffer at pH6˜8.

An embodiment of the luminescence-based recipe for the measurement of glucose comprises 0.1˜10 mM of luminol, 0.01˜500 U/mL of horse redish peroxidase (HRP), 0.01˜500 U/mL of glucose oxidase (GOx), 0˜10 mM of PIP, 0˜1% of Triton X-100, 0˜20 mM of EDTA, and 5˜200 mM of buffer at pH6˜8.

An embodiment of the luminescence-based recipe for the measurement of uric acid comprises 0.1˜10 mM of luminol, 0.01˜500 U/mL of HRP, 0.01˜500 U/mL of uricase, 0˜10 mM of PIP, 0˜1% of Triton X-100, 0˜20 mM of EDTA, and 5˜200 mM of buffer at pH6˜8.

A device using the above recipes is also provided. The device comprises a centrifugal unit and a luminescence analyzer and is characterized in that the centrifugal unit includes a rotary cylinder, and a rotation motor for exerting a centrifugal force for the rotary cylinder, wherein the rotary cylinder includes an inner surface and an interconnected outer surface, the outer surface has a plurality of radially extended openings, wherein the luminescence analyzer is disposed corresponding to one of the extended openings.

### BRIEF DESCRIPTION OF THE DRAWINGS

Embodiments of the invention can be more fully understood and further advantages become apparent when reference is made to the following description and the accompanying drawings in which:

FIG. 1A illustrates the relation between luminescence and the loading time of creatinine during the measurement of creatinine.

FIG. 1B illustrates the relation between alteration of luminescent intensity and loading concentration of creatinine.

FIG. 2A illustrates the relation between luminescent intensity and time in the presence of creatinine at different concentration during the measurement of creatinine in solution form.

FIG. 2B illustrates the calibration curve of the measurement of creatinine in solution form. The relative intensity was calculated by the sum area of normalized luminescent intensity as shown in FIG. 2A. The time interval is 50-90 sec.

FIG. 3A illustrates the relation between luminescent intensity and time in the presence of creatinine at different concentration during the measurement of creatinine in lyophilized form.

FIG. 3B illustrates the calibration curve of the measurement of creatinine in lyophilized form. The relative intensity was calculated by the sum area of normalized luminescent intensity as shown in FIG. 3A. The time interval is 20-60 sec.

FIG. 4A illustrates the relation between sum area and time in the presence of creatinine at different concentration during the measurement of serum creatinine in lyophilized form.

FIG. 4B illustrates the calibration curve of the measurement of serum creatinine. The relative intensity was calculated by the sum area of normalized luminescent intensity as shown in FIG. 4A. The time interval is 0-200 sec.

FIG. 5A illustrates the relation between luminescent intensity and time in the presence of urea at different concentration during the measurement of urea in solution form.

FIG. 5B illustrates the calibration curve of the measurement of urea in solution form. The relative intensity was calculated by the sum area of normalized luminescent intensity as shown in FIG. 5A. The time interval is 60-70 sec.

FIG. 6A illustrates the relation between luminescent intensity and time in the presence of urea at different concentration during the measurement of urea in lyophilized form.

FIG. 6B illustrates the calibration curve of the measurement of urea in lyophilized form. The relative intensity was calculated by the sum area of normalized luminescent intensity as shown in FIG. 6A. The time interval is 130-180 sec.

FIG. 7A illustrates the relation between normalized luminescent intensity and time in the presence of urea at different concentration during the measurement of serum urea in lyophilized form.

FIG. 7B illustrates the calibration curve of the measurement of serum urea in lyophilized form. The relative intensity was calculated by the sum area of normalized luminescent intensity as shown in FIG. 7A. The time interval is 0-60 sec.

FIG. 8A illustrates the relation between luminescent intensity and time in the presence of uric acid at different concentration during the measurement of uric acid in solution form.

FIG. 8B illustrates the calibration curve of the measurement of uric acid in solution form. The relative intensity was calculated by the sum area of the time interval 0-6 min (method 1). If the relative intensity is lower than 400, the normalized intensity is divided by the slope area of time interval 10-30 sec to provide a better solution for uric acid at low concentration (method 2) as shown in the scale drawing of FIG. 8B.

FIG. 9A illustrates the relation between luminescent intensity and time in the presence of uric acid at different concentration during the measurement of uric acid in lyophilized form.

FIG. 9B illustrates the calibration curve of the measurement of uric acid in lyophilized form. The relative intensity was calculated by the peak height of luminescent intensity as shown in FIG. 9A. The time interval is 0-10 min.

FIG. 10A illustrates the relation between sum area and time in the presence of uric acid at different concentration during the measurement of serum uric acid in lyophilized form.

FIG. 10B illustrates the adjusted curve of the measurement of serum uric acid in lyophilized form. The relative intensity was calculated by the sum area of time interval 0-5 min.

FIG. 11A illustrates the relation between luminescent intensity and time in the presence of glucose at different concentration during the measurement of glucose in solution form.

FIG. 11B illustrates the calibration curve of the measurement of glucose in solution form. The relative intensity was calculated by the sum area of normalized intensity as shown in FIG. 11A. The time interval is 0-3 min.

FIG. 12A illustrates the relation between luminescent intensity and time in the presence of glucose at different concentration during the measurement of glucose in lyophilized form.

FIG. 12B illustrates the calibration curve of the measurement of glucose in lyophilized form. The relative intensity was calculated by the peak height of luminescent intensity as shown in FIG. 12A. The time interval is 0-2 min.

FIG. 13A illustrates the relation between sum area and time in the presence of glucose at different concentration during the measurement of serum glucose in lyophilized form.

FIG. 13B illustrates the calibration curve of the measurement of serum glucose in lyophilized form. The relative intensity was calculated by the sum area of time interval 0-30 sec.

FIG. 14 illustrates an embodiment of the miniaturized luminescent analyzer of the example.

### DETAILED DESCRIPTION

Luminescence-based recipes and device using the same are provided.

Luminescence assay has sensitivity of hundred or thousand times than spectroscopic or calorimetric assays and is relatively easy in manipulation. In particular, most physiological markers or metabolites can be measured by luminescence assay. Luminescence can be, therefore, used in the development of fast analysis platform. Luminescence emission is produced when an electron falls from an excited state which is induced by chemical or biological reaction to a ground state. Luminescence emission can be classified as chemiluminescence and bioluminescence. The mechanism of the reactions is shown as below.

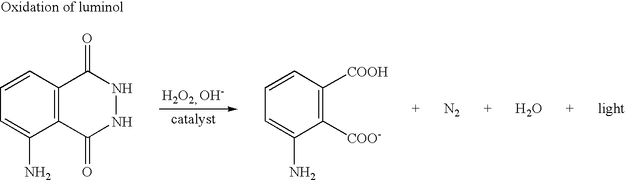

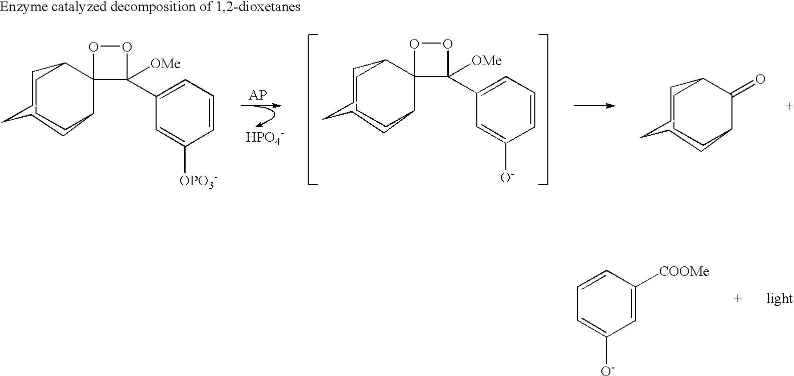

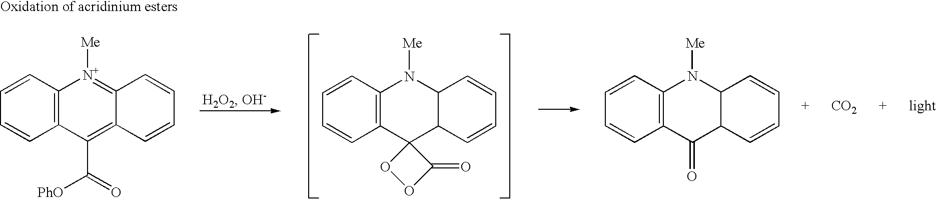

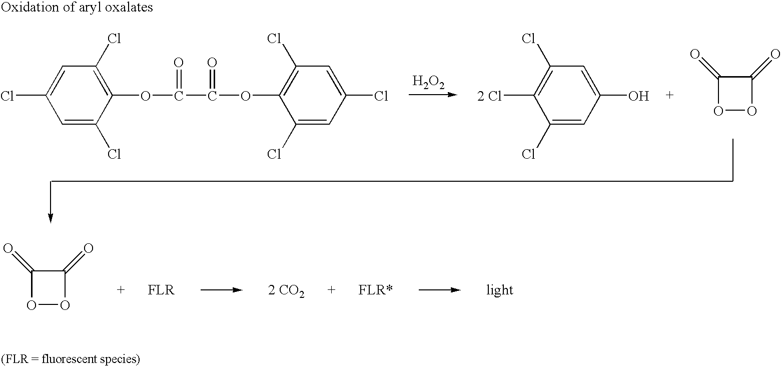

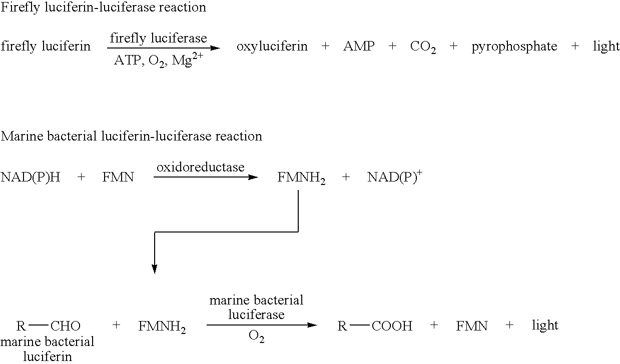

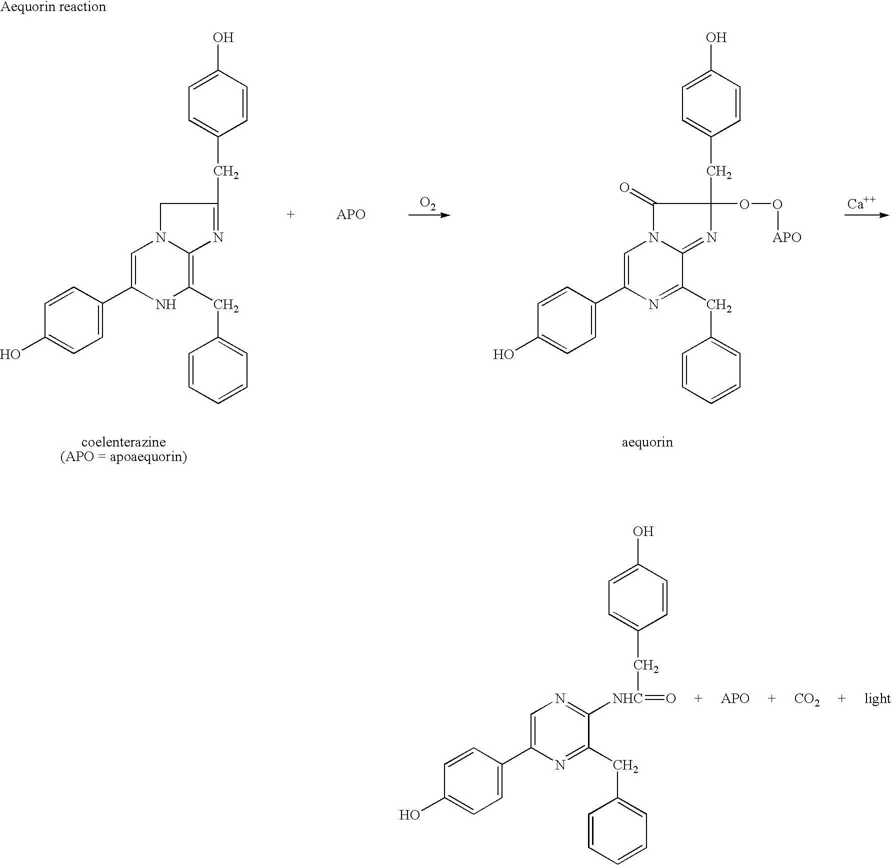

Chemiluminescence utilizes compounds such as luminol, 1,2-dioxetane, acridinium esters, and oxalate esters, or their derivatives where luminol is the most common. The emission mechanism of luminol is the oxidation in the presence of peroxides, usually hydrogen peroxide with an emission length of 450 nm. The reaction can be catalyzed by enzymes such as horseradish peroxidase, micro-peroxidase, catalase, or other substances such as hemoglobin, cytochrome c, Fe(III), and other metal complexes. The emission can be amplified by enhancers such as phenols, naphthols, amines to promote the sensitivity. Bioluminescence includes firefly luciferase, bacteria luciferase, and aequorin. Among these, luciferin-luciferase derived from firefly and marine bacteria are well-known and are with emission length of 580 nm and 450 nm respectively. Accordingly, chemiluminescence analysis is applied for analysts related to oxidation-reduction reaction, and bioluminescence analysis is applied for analysts related to ATP or NAD(P) reaction. One detector is adequate for varies reactions since the emission is in the range of visible light. In addition, these reactions are the most important mechanism for various enzyme-substrate reactions and can be applied in a wide field. Related application has been reported, for example, Rauch et al discloses a chemiluminescent assay using flow injection analysis system with luminol for the detection of choline or phospholipase D; Michel et al. discloses a three-enzyme detection system using bacteria luciferase for the detection of D-sorbitol with sensitivity of 50 nM in 4-6 min; Eu et al. discloses a firefly luciferase system with ATP competition for the detection of galactose.

In addition to having high sensitivity, luminescence analysis does not require excitation light source, filter, or electrodes since it only detects photons. Moreover, background interference will not occur since no fluorescence is emitted. Luminescence analysis has wide dynamic range of up to 5 decades, significantly reducing the complexity of sample pretreatment. The analysis is appropriate for quick detection since the emission is completed in few seconds. Present luminometer adopts photomultiplier tube (PMT) or avalanche photodiode (APD) as the detector and is equipped with signal processing system and sample bearing device, which is relatively simple and suitable for miniaturization to achieve portable purpose.

Kidney is an important organ for excreting waste and redundant water. Urine production is a complicated excretion and resorption process for the balance of electrolytes and pH. In addition, kidney also produces hormones and vitamins for normal functions of a body. Acute and chronic renal failure is always of concern and the development of a portable detector for renal function is necessary.

The assessment of renal function can not be done with a single indicator, but needs multiple analysts. The indicators for clinical use now mainly include blood urea nitrogen (BUN) and creatinine. BUN is released by the catabolism of amino acids and 60% of BUN is excreted by kidney. The increase of BUN indicates abnormal protein metabolism, acute glomerulonephritis, uremia, or urinary obstruction. The increase of creatinine may due to severe muscular disease, nephritis, or hyperthyroidism. The amount of creatinine in urine may provide representative creatinine clearance for the calculation of glomerular filtration rate (GFR) (Tietz N. W. (1970) Fundamentals of Clinical Chemistry, W.B. Sauders Company: USA, 5th edition, pp. 419). In addition, uric acid is the final product of the metabolism of purine and the increase of uric acid in blood indicates gout, renal failure, dehydration, metabolic acidosis, or excess purine uptake. A study from Australia shows that diabetics especially type II has 36% possibility of renal complication. Except for blood glucose control, monitoring of blood pressure and renal function is also important for diabetics in order to prevent diabetic nephropathy (Macisaac R. J., et al. (2004) Nonalbuminuric Renal Insufficiency in Type 2 Diabetes. Diabetes Care. 27, 195-200). Accordingly, the simultaneous detection of BUN, creatinine, uric acid, and glucose is helpful for the evaluation and monitoring of renal function. Table 1 shows the normal range, analysis mechanism, detection time, and physiological significance of the four analysts.

TABLE 1
Normal range, detection method, and clinical significance of urea, creatinine, uric acid, and glucose

| | | | | | Manual | | Automated | | |
|---|---|---|---|---|---|---|---|---|---|
| | Expected value¹ | | Analysis¹ | | manipulation¹ | | analyzer² | | |
| | Serum | Urine | | Abs. | Sample | Time | Sample | Time | Clinical |
| Target | (mg/dl) | (g/24 hrs) | mechanism | (nm)³ | amount (μL) | (min) | amount (μL) | (min) | significance |
| Urea | 10-50 | 20-35 | Urease/GLDH | 340 | 10 | 5-10 | 5 | <0.5 | Kidney function |
| Creatinine | Male: 0.6-1.2 | 1-1.5 | Jaffe | 510 | 100 | 2 | 20 | <0.5 | Kidney function |
| | Female: 0.5-1.0 | | | | | | | | |
| Uric acid | Male: 3.5-7.0 | 0.25-0.75 | Uricase/POD | 546 | 50 | 5-10 | 10 | <0.5 | Gout |
| | Female: 2.4-5.7 | | | | | | | | |
| Glucose | 60-110 | <0.5 | GOD/POD | 510 | 20 | 5-10 | 5 | <0.5 | Type II Diabetes |

¹obtained from Chema Diagnostica.
²estimated from the performance of Boehringer Mannheim/Hitachi 917 analyzer.
³Detection range (sensitivity) and standard deviation are Abs. = 10⁻³ and 10%, respectively. The exact sensitivity could be worse and limited.

The inventors developed luminescence-based recipes for the measurement of creatinine, urea, uric acid, or glucose with one-step reaction. The recipes can be used in an aqueous solution or lyophilized powder and are the most appropriate formula for the detection of trace analysts in a few amounts of sample with a stable and reliable sensitivity and a wide detection range.

Accordingly, one embodiment of the luminescence-based recipe for the measurement of creatinine includes 0.01˜150 U/mL of creatininase, 0.01˜150 U/mL of creatine kinase, 1×10⁻⁶˜5×10⁻² mg/mL of firefly luciferase, 0.1˜5000 μM of luciferin, 1 μM˜20 mM of MgSO₄, 0.1˜5000 μM of ATP, 0˜1% of BSA, 0˜50 mM of DTT (1,4-dithioerythritol)), and 5˜200 mM of buffer at pH6˜8.

The other embodiment of the luminescence-based recipe for the measurement of creatinine in solution form includes 0.4˜75 U/mL of creatininase, 0.01˜75 U/mL of creatine kinase, 5×10⁻⁴˜2×10² mg/mL of firefly luciferase, 5˜2000 μM of luciferin, 1 μM˜500 μM of MgSO₄, 0.5˜1000 μM of ATP, 0˜0.5% of BSA, 0˜40 mM of DTT, and 25˜50 mM of buffer at pH6˜8.

Another embodiment of the luminescence-based recipe for the measurement of creatinine in lyophilized form includes 5˜100 U/mL of creatininase, 5˜100 U/mL of creatine kinase, 0.1˜3×10⁻² 2 mg/mL of firefly luciferase, 5˜2000 μM of luciferin, 1 μM˜500 μM of MgSO₄, 0.51000 μM of ATP, 0˜0.5% of BSA, 0˜40 mM of DTT, and 25˜50 mM of buffer at pH6˜8.

Yet another embodiment of the luminescence-based recipe for the measurement of serum creatinine in lyophilized form includes 0.4˜75 U/mL of creatininase, 0.01˜75 U/mL of creatine kinase, 0.1˜3×10⁻² mg/mL of firefly luciferase, 5˜2000 μM of luciferin, 1 μM˜500 μM of MgSO₄, 0.5˜1000 μM of ATP, 0˜0.5% of BSA, 0˜40 mM of DTT, and 25˜50 mM of buffer at pH6˜8.

The buffer used for the luminescence-based recipe for the measurement of creatinine includes, but is not limited to, Gly-gly buffer, HEPES, Tris, Bis-Tris, Bis-Tris propane, MOPS, PIPES, phosphate, or borate, preferably Gly-gly buffer at pH 7.5.

One embodiment of the luminescence-based recipe for the measurement of urea includes 0.01˜100 U/mL of urea amidolyase (URL), 1×10⁻⁶˜5×10⁻² mg/mL of firefly luciferase, 0.1˜5000 μM of luciferin, 1 μM˜20 mM of MgSO₄, 0.1˜5000 μM of ATP, 0˜100 mM of KCl, 0˜100 mM of NaHCO₃, 0˜20 mM of EGTA, 0˜1% of BSA, 0˜50 mM of DTT (1,4dithioerythritol)), and 5˜200 mM of buffer at pH6˜8.

The other embodiment of the luminescence-based recipe for the measurement of urea in solution form includes 0.1˜50 U/mL of urea amidolyase (URL), 5×10⁻⁴˜2×10⁻² mg/mL of firefly luciferase, 5˜2000 μM of luciferin, 1 μM˜5 mM of MgSO₄, 0.5˜1000 μM of ATP, 0˜40 mM of KCl, 0˜40 mM of NaHCO₃, 010 mM of EGTA, 0˜0.5% of BSA, 0˜40 mM of DTT (1,4-dithioerythritol)), and 25˜50 mM of buffer at pH6˜8.

Another embodiment of the luminescence-based recipe for the measurement of urea in lyophilized form includes 0.1˜50 U/mL of urea amidolyase (URL), 0.1˜3×10⁻² mg/mL of firefly luciferase, 5˜2000 μM of luciferin, 1 μM˜5 mM of MgSO₄, 0.5˜3000 μM of ATP, 0˜40 mM of KCl, 0˜40 mM of NaHCO₃, 010 mM of EGTA, 0˜0.5% of BSA, 0˜40 mM of DTT (1,4-dithioerythritol)), and 25˜50 mM of buffer at pH6˜8.

Yet another embodiment of the luminescence-based recipe for the measurement of serum urea in lyophilized form includes 0.1˜50 U/mL of urea amidolyase (URL), 0.1˜3×10⁻² mg/mL of firefly luciferase, 5˜2000 μM of luciferin, 1 μM˜5 mM of MgSO₄, 0.5˜1000 μM of ATP, 0˜40 mM of KCl, 0˜40 mM of NaHCO₃, 0˜10 mM of EGTA, 0˜0.5% of BSA, 0˜40 mM of DTT (1,4-dithioerythritol)), and 25˜50 mM of buffer at pH6˜8.

The buffer used for the luminescence-based recipe for the measurement of urea includes, but is not limited to, Gly-gly buffer, HEPES, Tris, Bis-Tris, Bis-Tris propane, MOPS, PIPES, phosphate, or borate, preferably Gly-gly buffer at pH 7.5.

One embodiment of the luminescence-based recipe for the measurement of glucose includes 0.1˜10 mM of luminol, 0.01˜500 U/mL of horse redish peroxidase (HRP), 0.01˜500 U/mL of glucose oxidase (GOx), 0˜10 mM of PIP, 0˜1% of Triton X-100, 0˜20 mM of EDTA, and 5˜200 mM of buffer at pH6˜9.

The other embodiment of the luminescence-based recipe for the measurement of glucose in solution form includes 0.1˜5 mM of luminol, 0.01˜10 U/mL of horse redish peroxidase (HRP), 0.1˜200 U/mL of glucose oxidase (GOx), 0˜2 mM of PIP, 0˜0.1% of Triton X-100, 0˜5 mM of EDTA, and 25˜50 mM of buffer at pH6˜9.

Another embodiment of the luminescence-based recipe for the measurement of glucose in lyophilized form includes 0.1˜5 mM of luminol, 0.1˜10 U/mL of horse redish peroxidase (HRP), 0.1˜200 U/mL of glucose oxidase (GOx), 0˜2 mM of PIP, 0˜0.1% of Triton X-100, 0˜5 mM of EDTA, and 25˜50 mM of buffer at pH6˜9.

Yet another embodiment of the luminescence-based recipe for the measurement of serum glucose in lyophilized form includes 0.1˜5 mM of luminol, 0.1˜250 U/mL of horse redish peroxidase (HRP), 1˜200 U/mL of glucose oxidase (GOx), 0˜2 mM of PIP, 0˜0.1% of Triton X-100, 0˜5 mM of EDTA, and 25˜50 mM of buffer at pH6˜9.

The buffer used for the luminescence-based recipe for the measurement of glucose includes, but is not limited to, Gly-gly buffer, HEPES, Tris, Bis-Tris, Bis-Tris propane, MOPS, PIPES, phosphate, or borate, preferably Gly-gly buffer at pH 7.5.

One embodiment of the luminescence-based recipe for the measurement of uric acid includes 0.1˜10 mM of luminol, 0.01˜500 U/mL of HRP, 0.01˜500 U/mL of uricase, 010 mM of PIP, 0˜1% of Triton X-100, 0˜20 mM of EDTA, and 5˜200 mM of buffer at pH6˜9.

The other embodiment of the luminescence-based recipe for the measurement of uric acid in solution form includes 0.1˜5 mM of luminol, 0.01˜20 U/mL of HRP, 0.1˜100 U/mL of uricase, 0˜2 mM of PIP, 0˜0.1% of Triton X-100, 0˜5 mM of EDTA, and 25˜100 mM of buffer at pH6˜9.

Another embodiment of the luminescence-based recipe for the measurement of uric acid in lyophilized form includes 0.1˜5 mM of luminol, 0.1˜10 U/mL of HRP, 0.1˜100 U/mL of uricase, 0˜2 mM of PIP, 0˜0.1% of Triton X-100, 0˜5 mM of EDTA, and 25˜50 mM of buffer at pH6˜9.

Yet another embodiment of the luminescence-based recipe for the measurement of serum uric acid in lyophilized form includes 0.1˜5 mM of luminol, 0.1˜200 U/mL of HRP, 0.1˜100 U/mL of uricase, 0˜2 mM of PIP, 0˜0.1% of Triton X-100, 0˜5 mM of EDTA, and 25˜50 mM of buffer at pH6˜9.

The buffer used for the luminescence-based recipe for the measurement of uric acid includes, but is not limited to, Gly-gly buffer, HEPES, Tris, Bis-Tris, Bis-Tris propane, MOPS, PIPES, phosphate, or borate, preferably Gly-gly buffer at pH 8.5.

A device using the above recipes is also provided. The device comprises a centrifugal unit and a luminescence analyzer and is characterized in that the centrifugal unit includes a rotary cylinder, and a rotation motor for exerting a centrifugal force for the rotary cylinder, wherein the rotary cylinder includes an inner surface and an interconnected outer surface, the outer surface has a plurality of radially extended openings, the luminescent unit is disposed corresponding to one of the extended openings.

The embodiment of the device provides quick analysis for multiple analytes and is miniaturized for testing small amounts of sample, easy manipulation, dynamic measurement, and simultaneous analysis.

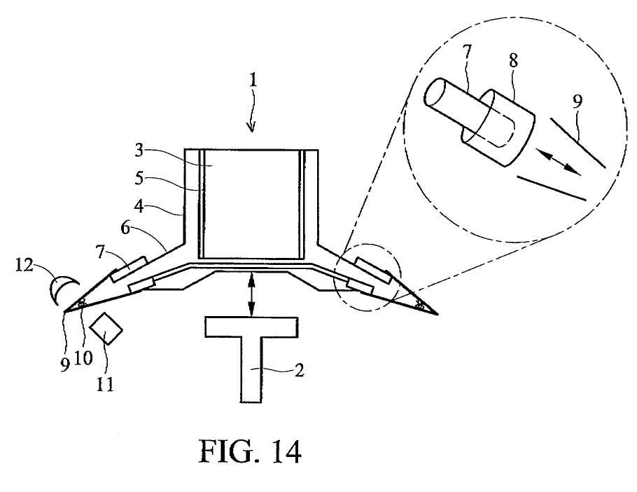

FIG. 14 illustrates one embodiment of the device of the invention. The device 1 comprises a centrifugal unit and a luminescence-based analyzer. Centrifuged serum sample is collected in a tube containing lyophilized recipes for luminescence detection immediately. The centrifugal unit includes a rotary cylinder (4 and 5), and a rotation motor 2 for exerting a centrifugal force for the rotary cylinder. The rotary cylinder includes an inner surface 3 and an interconnected outer surface 4, and the outer surface 4 has a plurality of radially extended openings, the separation parts 6. The inner surface 3 is covered with a disposable film 5 for the separation of blood cells and serum in whole blood. Blood cells are retained in the rotary cylinder and serum may pass through the film 5 to the separation parts 6. The disposable film 5 is acetate cellulose, nitrate cellulose, Polyethersulfone (PES), or polyvinylpyrrolidone (PVP). The opening of the separation part 6 is connected to a testing tube 9 for receiving centrifuged sera. A casing 8 may be disposed between the separation part 6 and the testing tube 9 for a close connection as illustrated in the partial enlargement of FIG. 14. The luminescence-based analyzer includes a incident light illuminator 11 and a emitted light receiver 12, disposed corresponding to the recipes 10 containing testing tube 9 for the detection of the luminescent light emitted from the serum sample.

Practical examples are described herein.

### EXAMPLES

Materials and Methods

Materials:

Table 2 shows chemicals used in the examples.

TABLE 2

| Type | Chemicals | Supplier |
|---|---|---|
| General | ATP | Sigma |
| | Luciferin | Sigma |
| | Firefly luciferase | Promega |
| | MgSO₄ | Sigma |
| | BSA | Sigma |
| | Gly-gly | Sigma |
| Creatinine | Creatinine | Sigma |
| | Creatininase | Toyobo |
| | Creatine kinase | Sigma |
| Urea | urea amidolyase (URL) | Toyobo |
| | KCl | RDH |
| | NaHCO₃ | Sigma |
| | EGTA | Sigma |
| General | Luminol | Sigma |
| | MgSO₄ | Sigma |
| | BSA | Sigma |
| | 4-indophenol | Aldrich |
| | HRP | Sigma |
| | Triton X-100 | Sigma |
| | EDTA | J. T. Baker |
| | Gly-gly | Sigma |
| Glucose | Glucose | RDH |
| | Glucose oxidase (GOx) | Sigma |
| Uric acid | Uric acid | Sigma |
| | Uricase | Sigma |

Equipments

A. Luminometer:

Type and factory: TD-20/20, Turner BioSystems

Setup: sensitivity is 47%.

Accessories: 8×50 mm testing tube(PP)

Or the luminescent analyzer of the invention.

B. Lyophilizer:

Type and factory: Heto Drywinner FD-3, JOUAN

Parameters: temperature of the compressor is −50° C. and vacuum is 0.5 hPa.

Procedures:

Preparation of the Embodiments of Chemiluminescent Recipes

The master mixture was prepared in accordance with the table listed below. Ten or twenty μl of the master mixture was placed into the testing tube. The master mixture was added into the sample and the RLU value was recorded at appropriate time by a luminometer when the test was performed in solution form. When the test was performed in lyophilized form, sample was added into the testing tube containing the lyophilized master mixture and the RLU value was recorded at appropriate time by the luminometer.

Lyophilization

The differences between blank and sample containing analytes should be determined by the master mixture. The determination of the master mixture was confirmed by the luminometer. Ten or twenty μl of the master mixture was placed into a testing tube and the solution was frozen in liquid nitrogen for 20 sec. The testing tube was placed in a Heto Drywinner for 6 hours for the lyophilization of the master mixture. The testing tube was then stored at 4° C. in dark.

The amount range of the analytes and algorithm thereof.

TABLE 3
analyte amounts and algorithm thereof

| | | | Analyte | | | |
|---|---|---|---|---|---|---|
| Analyte | Normal range | Type | conc.(mg/dl) | Algorithm | Figures | Recipes |
| Creatinine | 0.5-1.2 mg/dl | S | 0.1-11 | Sum area at 50-90 sec | 2A, 2B | Table4 |
| | (0.04-0.08 mM) | L | 0.6-11 | Sum area at 20-60 sec | 3A, 3B | Table5 |
| | | P | 0-22 | Sum area at 0-200 sec | 4A, 4B | Table6 |
| Urea | 10-50 mg/dl | S | 0-105 | Sum area at 60-70 sec | 5A, 5B | Table7 |
| | (3-15.2 mM) | L | 0-140 | slope different at 130-180 sec | 6A, 6B | Table8 |
| | | P | 0-20 | Sum area at 0-60 sec | 7A, 7B | Table9 |
| Uric acid | 2.4-7.0 mg/dl | S | 0.52-25 | Method 1: sum area at 0-6 min | 8A, 8B | Table10 |
| | (0.14-0.42 mM) | | | (If the relative intensity is | | |
| | | | | less than 400, Method 2 can be | | |
| | | | | applied.) | | |
| | | | | Method 2: sum area at 0-6 min | | |
| | | | | is divided by the slope at | | |
| | | | | 10-30 sec | | |
| | | L | 1.57-30 | Peak height at 0-10 min | 9A, 9B | Table11 |
| | | P | 0-27 | Sum area at 0-5 min | 10A, 10B | Table12 |
| Glucose | 60-110 mg/dl | S | 45-540 | Sum area at 0-3 min | 11A, 11B | Table13 |
| | (3.6-6.7 mM) | | | | | |
| | | L | 45-720 | Peak height at 0-2 min | 12A, 12B | Table14 |
| | | P | 45-800 | Sum area at 0-30 sec | 13A, 13B | Table15 |

S: determination of analyte in buffer by solution form
L: determination of analyte in buffer by lyophilization powder
P: determination of analyte in plasma by lyophilization powder

### Example 1

### Preliminary Experiment for the Measurement of Creatinine

Creatinine analysis is illustrated as an example. The mechanism of luminescence-based analysis includes three steps: (1) creatinine is converted to creatine by creatininase; (2) creatine is phosphorized by creatine kinase with consumption of ATP; (3) the concentration of creatinine in the system is determined by ATP competition compared with firefly luciferase-luciferin system. The reactions are shown as below.

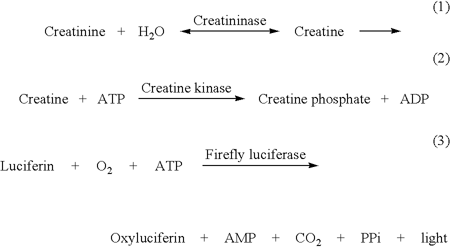

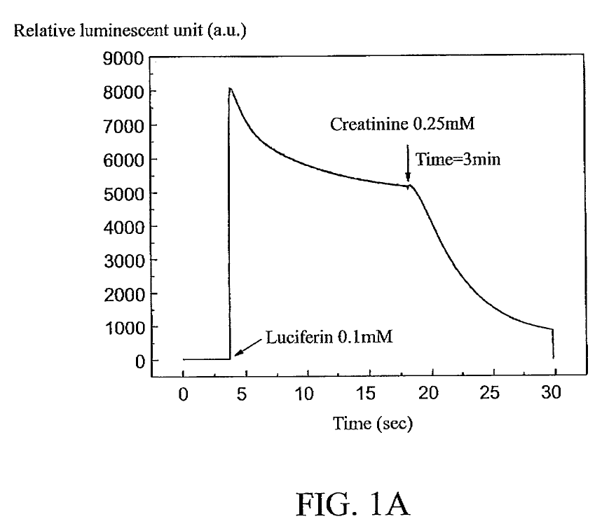

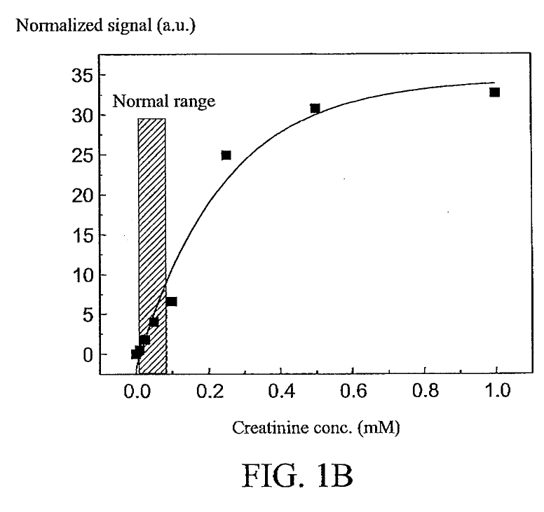

FIG. 1A illustrates the relation between luminescent signal and loading time. When firefly luciferase was added into the system, reaction (3) occurred immediately and the luminescence intensity increased rapidly. When the reaction was slowed down (for example, 3 minutes later), newly added creatinine lead to significant reduction of signal, indicating reactions (1) and (2) were carrying out. ATP in the system was consumed by these reactions in competition with reaction (3), and the curve became steep. FIG. 1B illustrates the relation between the concentration of creatinine to the changes of signal intensity. The results show that the detection range is 0˜1 mM. The normal range of creatinine in human body is between 0.04˜0.08 mM, and creatinine may become 1 mM when renal malfunction. This indicates that the system has good sensitivity and appropriate dynamic range. Pretreatment of sample is unnecessary for the system.

The present detector for multiple analytes may not be reliable when applied for sample in small amount volume, however, small amount of sample is the key point for portable detector. In addition, the detection ranges for different analytes are various, for example, the normal range for blood glucose is between 0.6˜6.7 mM, but for other analytes may be below sub-mM, for example, the normal range for uric acid is between 0.14˜0.42 mM. A broad detection range is, therefore, required in addition to micro-detection.

Sample obtained by blood taking needle can be tens μl of blood, and the reaction volume in the system is about 10 μl. The requirement of small amount sampling can be achieved by the embodiment of the system of the invention. The present biosensor for the detection of creatinine is mainly through electrochemical mechanism with the problem of weak sensitivity or serious matrix interference (Soldatkin A. P., et al. (2002) Creatinine sensitive biosensor based on ISFETs and creatinine deiminase immobilized in BSA membrane. Talanta 58, 352-357) and cannot be applied in practice. In the contrary, the embodiment of the system of the invention overcomes these problems is shown in FIGS. 1A and 1B.

The detection of creatinine, urea, uric acid, and glucose is shown below.

### Example 2

### Detection of Creatinine

Detection of Creatinine in Solution Form

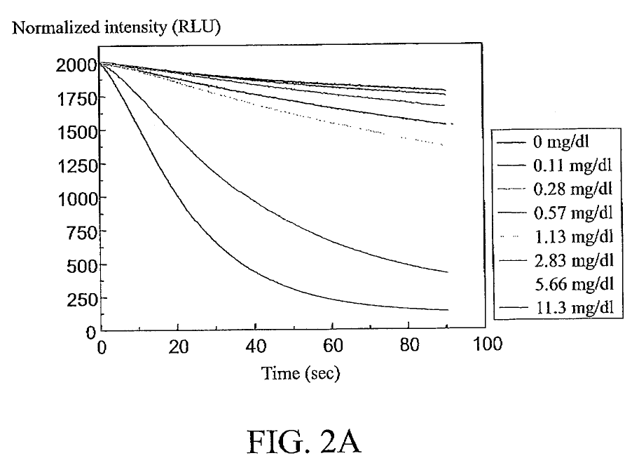

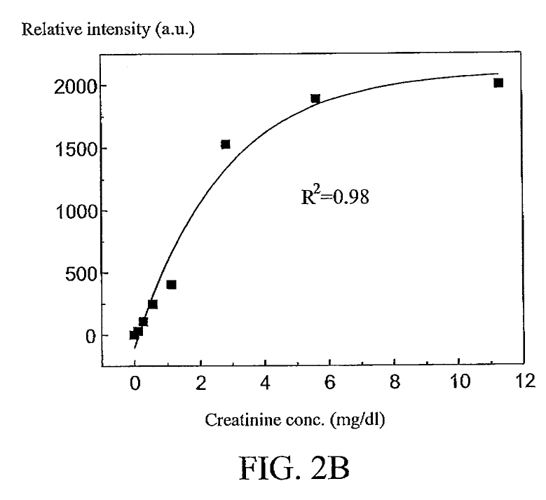

The detection was performed according to the materials and methods as described above and the recipes listed in table 4. Twenty μL of the master mixture was added to each tube and 1 μL of creatinine solution was introduced. The results are shown as FIGS. 2A and 2B.

TABLE 4
chemical recipes for the detection of creatinine in solution form

| | Stock | | Running | | Amount |
|---|---|---|---|---|---|
| Chemicals | solution | | conc. | | (μL) |
| Gly-gly buffer at pH 7.5 | 1M | | 50 | mM | 10 |
| MgSO₄ | 5 | mM | 10 | μM | 0.8 |
| BSA | 5% | | 0.1% | | 8 |
| ATP | 0.1 | mM | 0.5 | μM | 2 |
| Creatininase | 5000 | U/mL | 75 | U/mL | 6 |
| Creatine kinase | 7000 | U/mL | 75 | U/mL | 4.3 |
| Firefly luciferase | 14.4 | mg/mL | 1.4 × 10⁻² mg/mL | | 0.4 |
| Luciferin | 2 | mM | 0.1 | mM | 20 |

| | | | |
|---|---|---|---|
| H₂O | — | — | 349 |

| | |
|---|---|
| Total amount | 400 |

FIG. 2A illustrates the relation between luminescent intensity and time at the presence of creatinine in different concentration. Creatinine was prepared in 25 mM of Gly-gly buffer at pH7.5. FIG. 2B illustrates the adjusted curve of the measurement of creatinine in solution form. The relative intensity was calculated by the sum area of normalized luminescent intensity as shown in FIG. 2A. The time interval is 50-90 sec.

B. Detection of Creatinine in Lyophilized Form

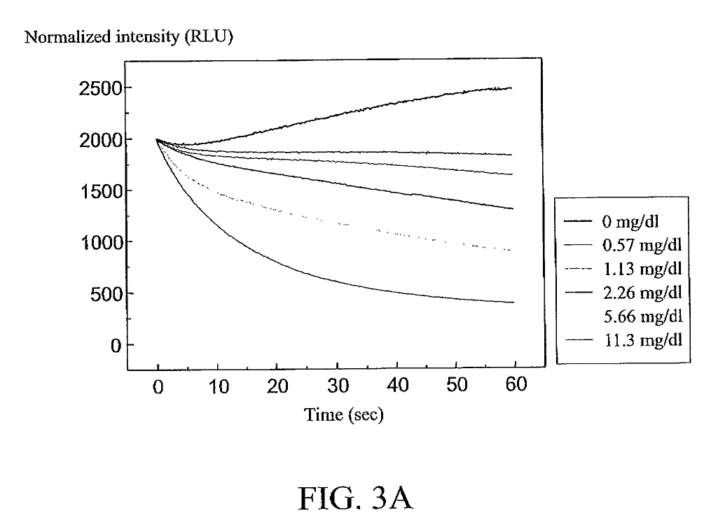

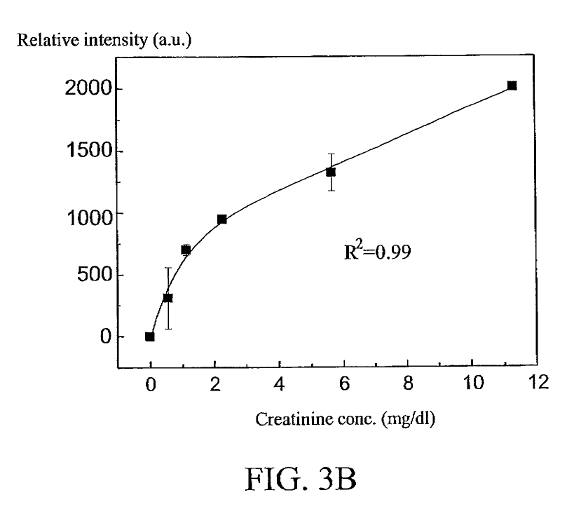

The detection was performed according to the materials and methods as above described and the recipes listed in table 5. Twenty μL of the master mixture was added to each tube and 20 μL of creatinine solution was introduced. The results are shown as FIGS. 3A and 3B.

TABLE 5
chemical recipes for the detection of creatinine in lyophilized form

| | Stock | | Running | | Amount |
|---|---|---|---|---|---|
| Chemicals | solution | | conc. | | (μL) |
| Gly-gly buffer at pH 7.5 | 1M | | 50 | mM | 10 |
| MgSO₄ | 5 | mM | 10 | μM | 0.8 |
| BSA | 5% | | 0.1% | | 8 |
| ATP | 0.1 | mM | 0.5 | μM | 2 |
| Creatininase | 5000 | U/mL | 75 | U/mL | 6 |
| creatine kinase | 7000 | U/mL | 75 | U/mL | 4.3 |
| Firefly luciferase | 14.4 | mg/mL | 1.4 × 10⁻² mg/mL | | 0.4 |
| luciferin | 2 | mM | 0.1 | mM | 20 |

| | | | |
|---|---|---|---|
| H₂O | — | — | 349 |

| | |
|---|---|
| Total amount | 400 |

FIG. 3A illustrates the relationship between luminescent intensity and time in the presence of creatinine at different concentration. Creatinine was prepared in 25 mM of Gly-gly buffer at pH7.5. FIG. 3B illustrates the adjusted curve of the measurement of creatinine in lyophilized form. The relative intensity was calculated by the sum area of normalized luminescent intensity as shown in FIG. 3A. The time interval is 20-60 sec.

C. Detection of Serum Creatinine in Lyophilized Form

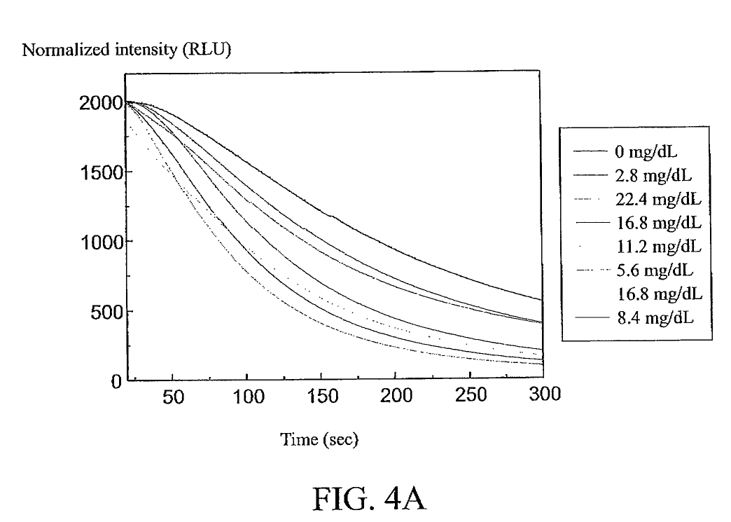

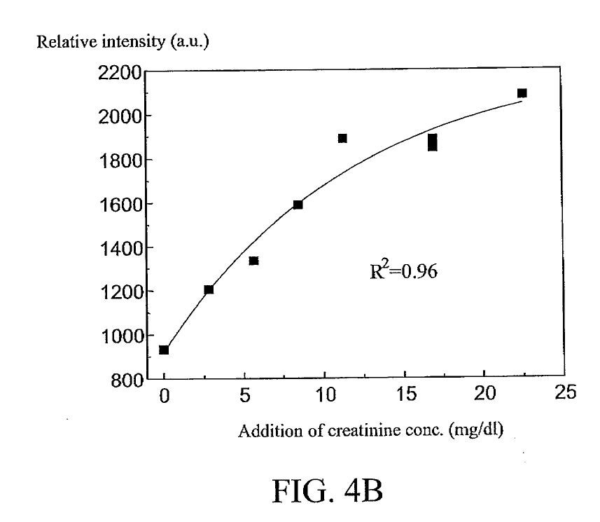

The detection was performed according to the materials and methods as above described and the recipes listed in table 6. Twenty μL of the master mixture was added to each tube and 20 μL of creatinine solution was introduced. The results are shown as FIGS. 4A and 4B.

TABLE 6
chemical recipes for the detection of
serum creatinine in lyophilized form

| | Stock | | Running | | Amount |
|---|---|---|---|---|---|
| Chemicals | solution | | conc. | | (μL) |
| Gly-gly buffer at pH 7.5 | 1M | | 50 | mM | 10 |
| MgSO₄ | 5 | mM | 10 | μM | 0.8 |
| BSA | 5% | | 0.1% | | 8 |
| ATP | 0.1 | mM | 7 | μM | 28 |
| Creatininase | 5000 | U/mL | 75 | U/mL | 6 |
| Creatine kinase | 7000 | U/mL | 75 | U/mL | 4.3 |
| Firefly luciferase | 14.4 | mg/mL | 1.4 × 10⁻² mg/mL | | 0.4 |
| Luciferin | 2 | mM | 0.1 | mM | 20 |

| | | | |
|---|---|---|---|
| H₂O | — | — | 323 |

| | |
|---|---|
| Total amount | 400 |

FIG. 4A illustrates the relationship between sum area and time in the presence of creatinine at different concentration. Creatinine was spiked in serum. FIG. 4B illustrates the adjusted curve of the measurement of serum spiked with creatinine. The relative intensity was calculated by the sum area of normalized luminescent intensity as shown in FIG. 4A. The time interval is 0-200 sec.

### Example 3

### Detection of Urea

A. Detection of Urea in Solution Form

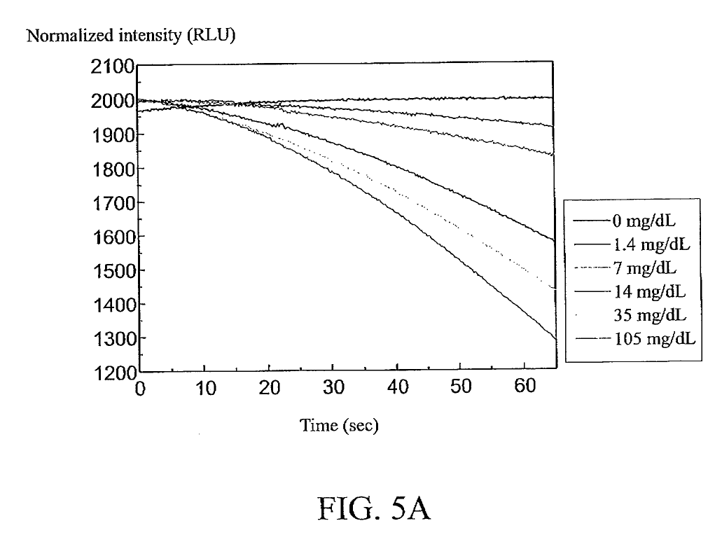

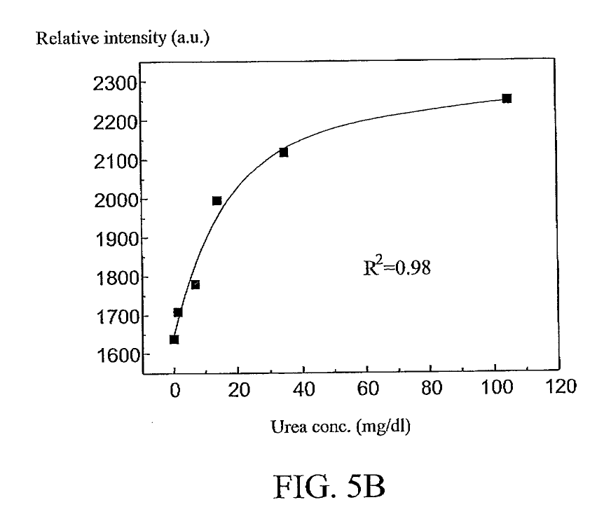

The detection was performed according to the materials and methods as above described and the recipes listed in table 7. Ten μL of the master mixture was added to each tube and 0.5 μL of urea solution was introduced. The results are shown as FIGS. 5A and 5B.

TABLE 7
chemical recipes for the detection of urea in solution form

| | Stock | | Running | | Amount |
|---|---|---|---|---|---|
| Chemicals | solution | | conc. | | (μL) |
| Gly-gly buffer at | 1M | | 25 | mM | 7.5 |
| pH 7.5 | | | | | |
| luciferin | 15 | mM | 2 | mM | 40 |
| BSA | 5% | | 0.1% | | 6 |
| KCl | 1M | | 30 | mM | 9 |
| NaHCO₃ | 1M | | 10 | mM | 3 |
| DTT | 1M | | 10 | mM | 3 |
| MgSO₄ | 1M | | 5 | mM | 1.5 |
| URL | 150 | U/mL | 2 | U/ml | 4 |
| firefly luciferase | 1.4 × 10⁻² mg/mL | | 1.4 × 10⁻³ mg/mL | | 30 |
| ATP | 15 | mM | 0.75 | mM | 15 |

| | | | |
|---|---|---|---|
| H₂O | — | — | 196 |

| | |
|---|---|
| Total amount | 270 |

FIG. 5A illustrates the relationship between luminescent intensity and time in the presence of urea at different concentration. Urea solution was prepared in 25 mM of Gly-gly buffer at pH7.5. FIG. 5B illustrates the adjusted curve of the measurement of urea in solution form. The relative intensity was calculated by the sum area of normalized luminescent intensity as shown in FIG. 5A. The time interval is 60-70 sec.

B. Detection of Urea in Lyophilized Form

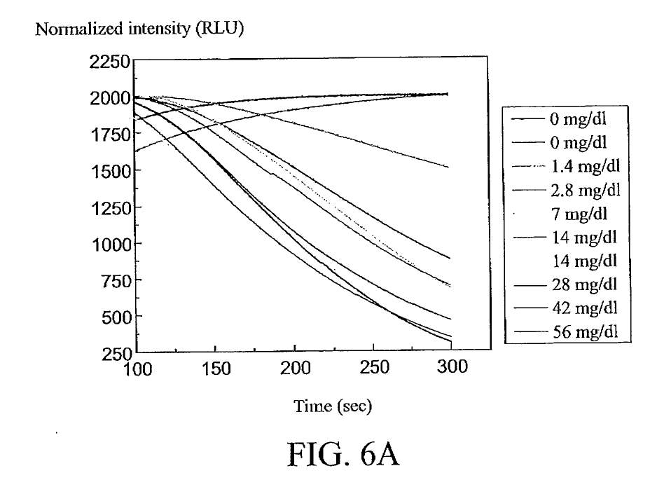

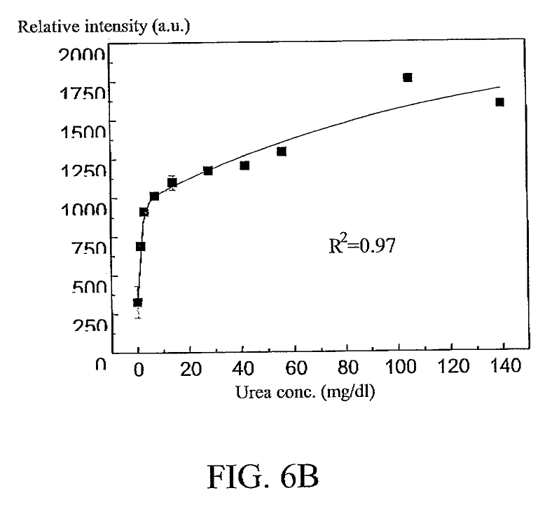

The detection was performed according to the materials and methods as described above and the recipes listed in table 8. Five μL of the master mixture was added to each tube for lyophilization and the tube was stored at 4° C. in dark. The results are shown as FIGS. 6A and 6B.

TABLE 8
chemical recipes for the detection of urea in lyophilized form

| | Stock | | Running | | Amount |
|---|---|---|---|---|---|
| Chemicals | solution | | conc. | | (μL) |
| Gly-gly buffer at | 1M | | 50 | mM | 7.5 |
| pH 7.5 | | | | | |
| luciferin | 15 | mM | 4 | mM | 40 |
| BSA | 5% | | 0.2% | | 6 |
| KCl | 1M | | 60 | mM | 9 |
| NaHCO₃ | 1M | | 20 | mM | 3 |
| DTT | 1M | | 20 | mM | 3 |
| MgSO₄ | 1M | | 10 | mM | 1.5 |
| URL | 150 | U/mL | 6 | U/mL | 6 |
| firefly luciferase | 1.4 × 10⁻² mg/mL | | 2.8 × 10⁻³ mg/mL | | 30 |
| ATP | 15 | mM | 1.5 | mM | 15 |

| | | | |
|---|---|---|---|
| H₂O | — | — | 29 |

| | |
|---|---|
| Total amount | 150 |

FIG. 6A illustrates the relationship between luminescent intensity and time in the presence of urea at different concentration during the measurement of urea in lyophilized form. Urea solution was prepared in 25 mM of Gly-gly buffer at pH7.5. FIG. 6B illustrates the adjusted curve of the measurement of urea in lyophilized form. The relative intensity was calculated by the slope difference of normalized luminescent intensity as shown in FIG. 6A. The time interval is 130-180 sec.

C. Detection of Serum Urea in Lyophilized Form

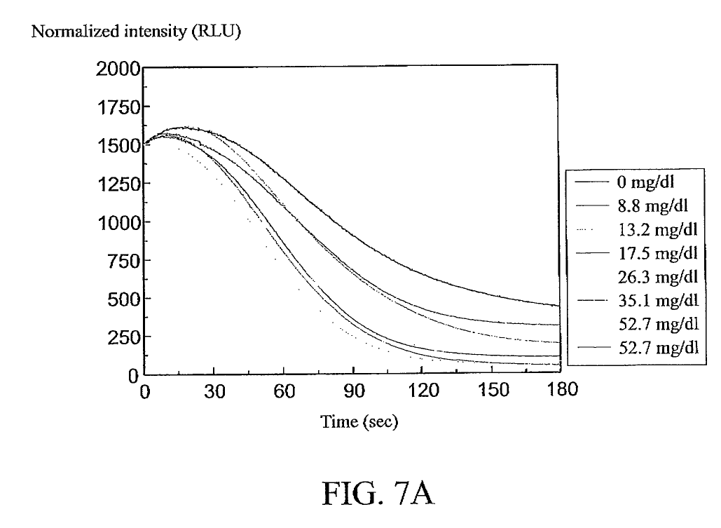

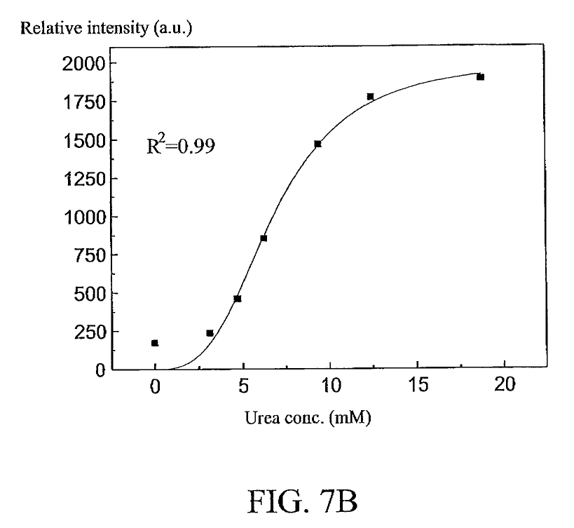

The detection was performed according to the materials and methods as described above and the recipes listed in table 9. Five μL of the master mixture was added to each tube for lyophilization and the tube was stored at 4° C. in dark. The results are shown as FIGS. 7A and 7B.

TABLE 9
chemical recipes for the detection of serum urea

| | Stock | | Running | | Amount |
|---|---|---|---|---|---|
| Chemicals | solution | | conc. | | (μL) |
| Gly-gly buffer at | 1M | | 50 | mM | 5 |
| pH 7.5 | | | | | |
| luciferin | 15 | mM | 4 | mM | 26.7 |
| BSA | 5% | | 0.5% | | 10 |
| KCl | 1M | | 60 | mM | 6 |
| NaHCO₃ | 1M | | 20 | mM | 2 |
| MgSO₄ | 1M | | 10 | mM | 1 |
| URL | 150 | U/mL | 12 | U/mL | 8 |
| firefly luciferase | 1.4 × 10⁻² mg/mL | | 2.8 × 10⁻³ mg/mL | | 20 |
| EGTA | 80 | mM | 8 | mM | 10 |
| ATP | 15 | mM | 1.5 | mM | 10 |

| | | | |
|---|---|---|---|
| H₂O | — | — | 1.3 |

| | |
|---|---|
| Total amount | 100 |

FIG. 7A illustrates the relationship between normalized luminescent intensity and time in the presence of urea with different concentration. Urea was spiked in serum. FIG. 7B illustrates the adjusted curve of the measurement of serum urea. The relative intensity was calculated by the sum area of normalized luminescent intensity as shown in FIG. 7A. The time interval is 0-60 sec.

### Example 4

### Detection of Uric Acid

A. Detection of Uric Acid in Solution Form

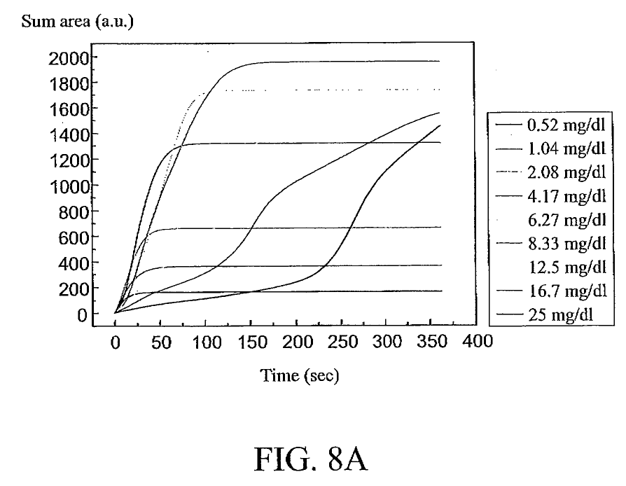

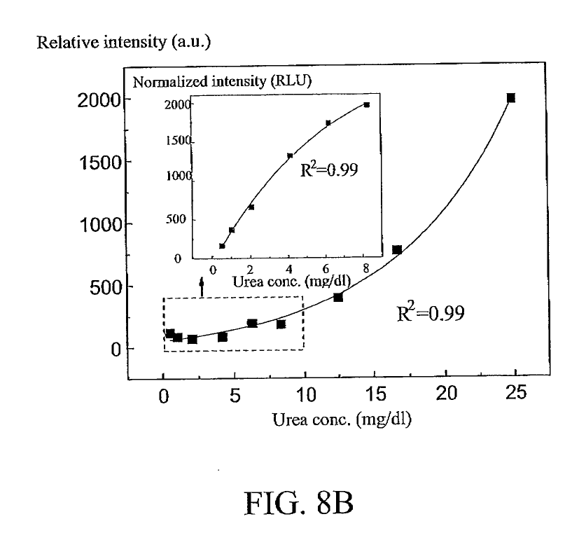

The detection was performed according to the materials and methods as above described and the recipes listed in table 10. Ten μL of the master mixture was added to each tube and 5 μL of uric acid solution was introduced. The results are shown as FIGS. 8A and 8B.

TABLE 10
chemical recipes for the detection of uric acid

| | Stock | | Running | | Amount |
|---|---|---|---|---|---|
| Chemicals | solution | | conc. | | (μL) |
| Gly-gly buffer at pH 8.5 | 1M | | 50 | mM | 10 |
| PIP | 50 | mM | 0.25 | mM | 1 |
| Luminol | 100 | mM | 0.5 | mM | 1 |
| HRP | 34.54 | U/mL | 0.41 | U/mL | 2.4 |
| Urease | 216 | U/mL | 47.5 | U/mL | 44 |
| Triton X-100 | 0.1% | | 0.001% | | 2 |
| EDTA | 500 | mM | 1 | mM | 0.4 |

| | | | |
|---|---|---|---|
| H₂O | — | — | 139.2 |

| | |
|---|---|
| Total amount | 200 |

FIG. 8A illustrates the relationship between luminescent intensity and time in the presence of uric acid at different concentration. Uric acid solution was prepared in 25 mM of Gly-gly buffer at pH8.5. FIG. 8B illustrates the adjusted curve of the measurement of uric acid in solution form. The relative intensity was calculated by the area summation of the time interval 0-6 min (method 1). If the relative intensity is less than 400, the normalized intensity is defined by summation of 0-6 min divided by slope between 10-30 sec. In this way, it can provide a better resolution for uric acid at low concentration (method 2) as shown in the small figure of FIG. 8B.

B. Detection of Uric Acid in Lyophilized Form

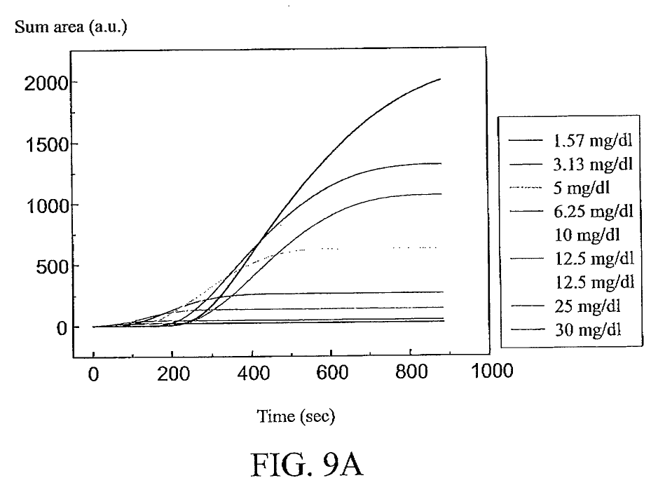

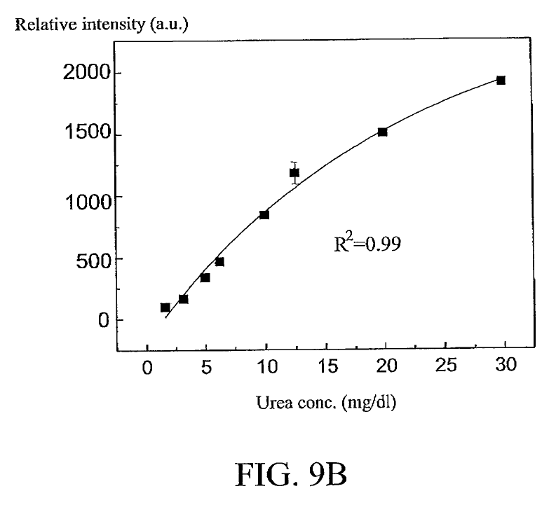

The detection was performed according to the materials and methods as described above and the recipes listed in table 11. Ten μL of the master mixture was added to each tube for lyophilization and 10 μL of uric acid solution was then introduced. The results are shown as FIGS. 9A and 9B.

TABLE 11
chemical recipes for the detection of uric acid in lyophilized form

| | Stock | | Running | | Amount |
|---|---|---|---|---|---|
| Chemicals | solution | | conc. | | (μL) |
| Gly-gly buffer at pH 8.5 | 1M | | 50 | mM | 10 |
| PIP | 50 | mM | 1 | mM | 4 |
| Luminol | 100 | mM | 2 | mM | 4 |
| BSA | 5% | | 0.25% | | 10 |
| HRP | 34.54 | U/mL | 0.3 | U/mL | 1.7 |
| Urease | 216 | U/mL | 47.5 | U/mL | 44 |
| Triton X-100 | 0.1% | | 0.001% | | 2 |
| EDTA | 500 | mM | 1 | mM | 0.4 |

| | | | |
|---|---|---|---|
| H₂O | — | — | 124 |

| | |
|---|---|
| Total amount | 200 |

FIG. 9A illustrates the relationship between luminescent intensity and time in the presence of uric acid at different concentration. Uric acid solution was prepared in 25 mM of Gly-gly buffer at pH8.5. FIG. 9B illustrates the normalized curve of the measurement of uric acid in lyophilized form. The relative intensity was calculated by the peak value of normalized luminescent intensity as shown in FIG. 9A. The time interval is 0-10 min/peak.

C. Detection of Serum Uric Acid in Lyophilized Form

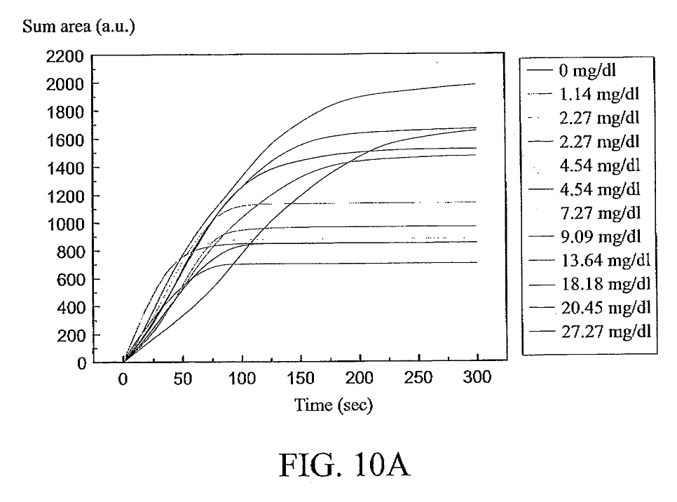

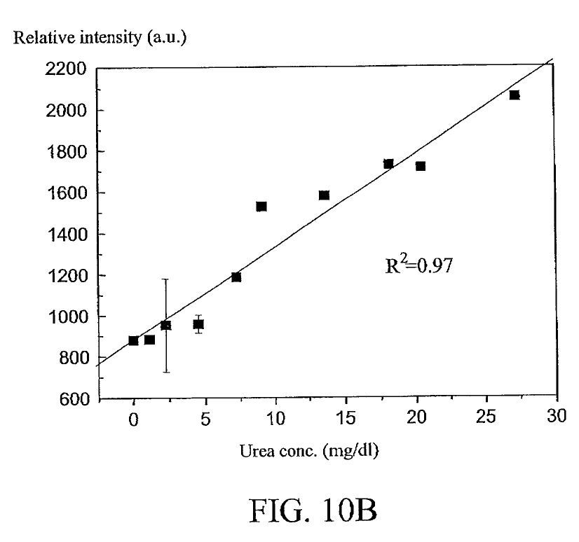

The detection was performed according to the materials and methods as described above and the recipes listed in table 12. Ten μL of the master mixture was added to each tube for lyophilization and 10 μL of serum spiked with different uric acid concentration was then introduced. The results are shown as FIGS. 10A and 10B.

TABLE 12
chemical recipes for the detection of
serum uric acid in lyophilized form

| | Stock | | Running | | Amount |
|---|---|---|---|---|---|
| Chemicals | solution | | conc. | | (μL) |
| Gly-gly buffer at pH 8.5 | 1M | | 50 | mM | 10 |
| PIP | 50 | mM | 1.25 | mM | 5 |
| Luminol | 100 | mM | 3 | mM | 6 |
| BSA | 5% | | 0.25% | | 10 |
| HRP | 34.54 | U/mL | 2.5 | U/mL | 14.5 |
| Urease | 216 | U/mL | 40 | U/mL | 37 |
| Triton X-100 | 0.1% | | 0.001% | | 2 |
| EDTA | 500 | mM | 1 | mM | 0.4 |

| | | | |
|---|---|---|---|
| H₂O | — | — | 115 |

| | |
|---|---|
| Total amount | 200 |

FIG. 10A illustrates the relationship between intensity and time in the presence of uric acid at different concentration. Uric acid was spiked in serum. FIG. 10B illustrates the adjusted curve of the measurement of serum uric acid. The relative intensity was calculated by the area summation of time interval 0-5 min.

### Example 5

### Detection of Glucose

A. Detection of Glucose in Solution Form

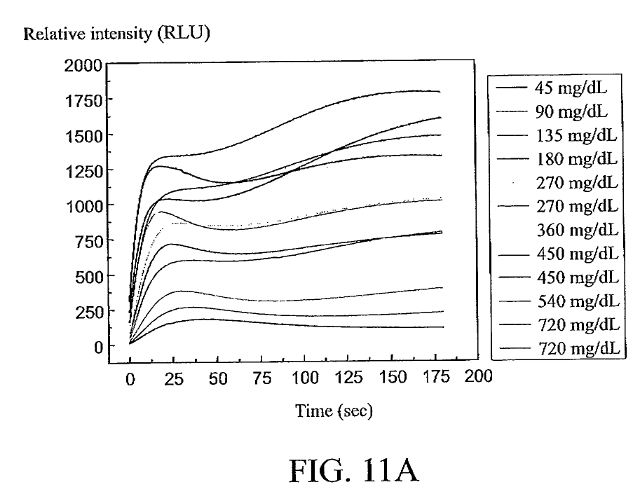

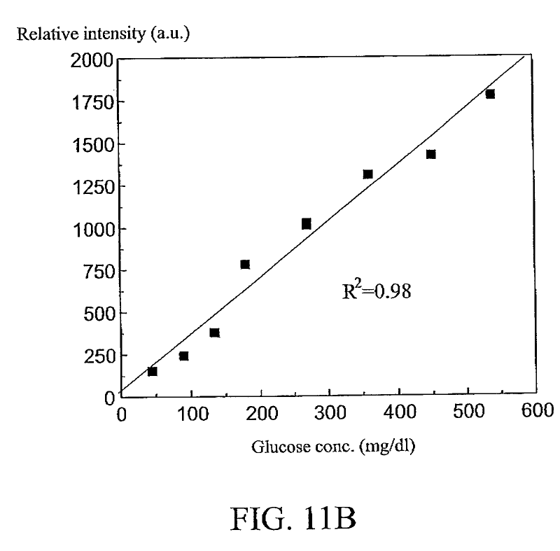

The detection was performed according to the materials and methods as described above and the recipes listed in table 13. Ten μL of the master mixture was added to each tube and 1 μL of glucose solution was then introduced. The results are shown as FIGS. 11A and 11B.

TABLE 13
chemical recipes for the detection of glucose in solution form

| | Stock | | Running | | Amount |
|---|---|---|---|---|---|
| Chemicals | solution | | conc. | | (μL) |
| Gly-gly buffer at pH 7.5 | 1M | | 50 | mM | 5 |
| PIP | 50 | mM | 0.5 | mM | 1 |
| Luminol | 100 | mM | 0.5 | mM | 0.5 |
| HRP | 34.54 | U/mL | 0.34 | U/mL | 1 |
| GOx | 110.4 | U/mL | 5 | U/mL | 4.5 |
| Triton X-100 | 0.1% | | 0.001% | | 1 |
| EDTA | 500 | mM | 1 | mM | 0.2 |
| H₂O | — | | — | | 87 |
| Total amount | | | | | 100 |

FIG. 11A illustrates the relationship between luminescent intensity and time in the presence of glucose at different concentration. Glucose solution was prepared in 25 mM of Gly-gly buffer at pH7.5. FIG. 11B illustrates the adjusted curve of the measurement of glucose in solution form. The relative intensity was calculated by the area summation of normalized intensity as shown in FIG. 11A. The time interval is 0-3 min.

B. Detection of Glucose in Lyophilized Form

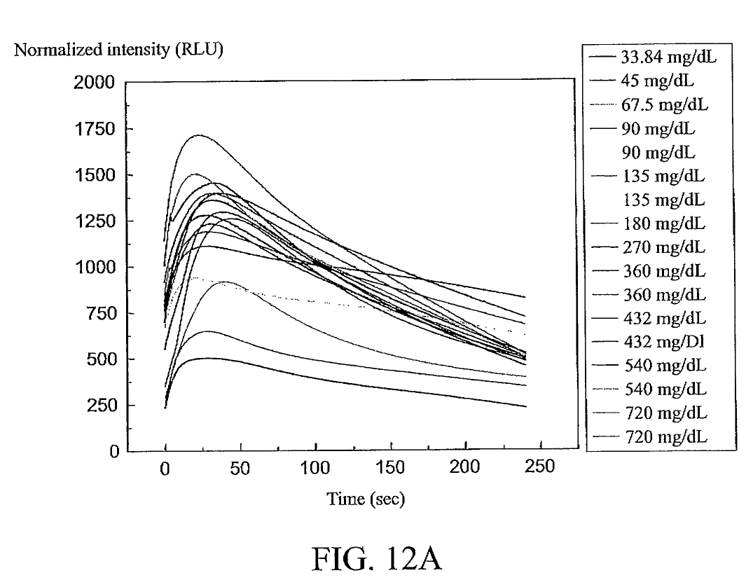

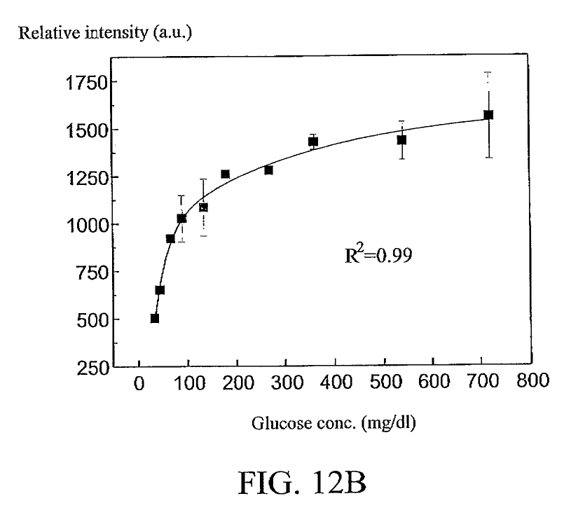

The detection was performed according to the materials and methods as above described and the recipes listed in table 14. Ten μL of the master mixture was added to each tube for lyophilization and 10 μL of glucose solution with different concentration was then introduced. The results are shown as FIGS. 12A and 12B.

TABLE 14
chemical recipes for the detection of glucose in lyophilized form.

| | Stock | | Running | | Amount |
|---|---|---|---|---|---|
| Chemicals | solution | | conc. | | (μL) |
| Gly-gly buffer at pH 7.5 | 1M | | 50 | mM | 10 |
| PIP | 50 | mM | 0.5 | mM | 2 |
| Luminol | 100 | mM | 0.5 | mM | 1 |
| HRP | 34.54 | U/mL | 0.34 | U/mL | 2 |
| GOx | 110.4 | U/mL | 5 | U/mL | 9 |
| Triton X-100 | 0.1% | | 0.001% | | 2 |
| EDTA | 500 | mM | 1 | mM | 0.4 |
| H₂O | — | | — | | 174 |
| Total amount | | | | | 200 |

FIG. 12A illustrates the relationship between luminescent intensity and time in the presence of glucose at different concentration. Glucose solution was prepared in 25 mM of Gly-gly buffer at pH7.5. FIG. 12B illustrates the adjusted curve of the measurement of glucose in lyophilized form. The relative intensity was calculated by the peak value of normalized luminescent intensity as shown in FIG. 12A. The time interval is 0-2 min.

C. Detection of Serum Glucose in Lyophilized Form

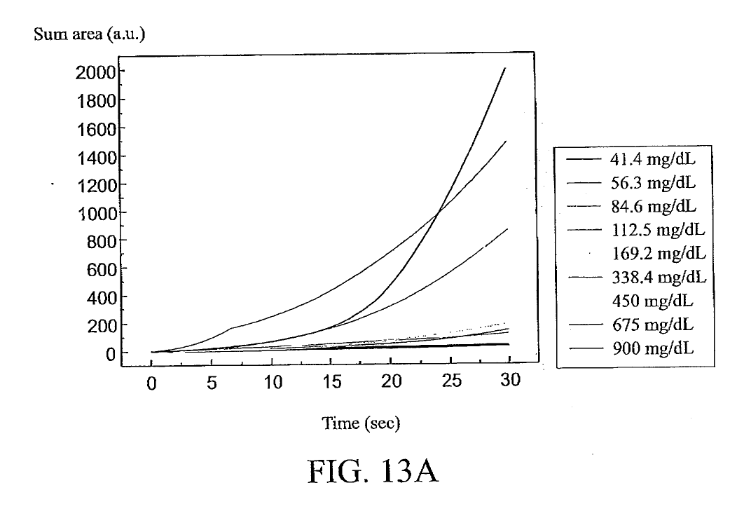

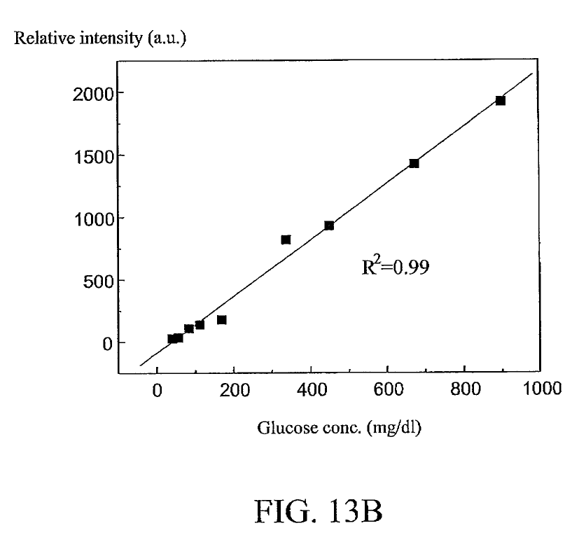

The detection was performed according to the materials and methods as described above and the recipes listed in table 15. Ten μL of the master mixture was added to each tube for lyophilization and 10 μL of glucose solution with different concentration was then introduced. The results are shown as FIGS. 13A and 13B.

TABLE 15
chemical recipes for the detection of serum glucose

| | | | |
|---|---|---|---|
| | Stock | Running | Amount |
| Chemicals | solution | conc. | (μL) |

| Gly-gly buffer at pH 7.5 | 1M | | 50 | mM | 10 |
|---|---|---|---|---|---|
| PIP | 50 | mM | 1 | mM | 4 |
| Luminol | 100 | mM | 1 | mM | 2 |
| HRP | 34.54 | U/mL | 2.5 | U/mL | 14.5 |
| GOx | 1104 | U/mL | 55 | U/mL | 10 |
| Triton X-100 | 0.1% | | 0.001% | | 2 |
| EDTA | 500 | mM | 1 | mM | 0.4 |
| H₂O | — | | — | | 157 |
| Total amount | | | | | 200 |

FIG. 13A illustrates the relation between intensity and time in the presence of glucose at different concentration. Glucose was spiked in serum. FIG. 13B illustrates the adjusted curve of the measurement of glucose spiked in serum. The relative intensity was calculated by the area summation of time interval 0-30 sec.

While the invention has been described by way of example and in terms of preferred embodiment, it is to be understood that the invention is not limited thereto.

What is claimed is:

1. A method for measuring urea, comprising:
obtaining a biological sample from a subject;
providing a luminescence-base composition for the measurement of urea, wherein the composition comprising a mixture of 0.01-100 U/mL of urea amidolyase (URL), 1×10⁻⁶-5×10⁻² mg/mL of firefly luciferase, 0.1-5000 μM of luciferin, 1 μM-20 mM of MgSO4, 0.1-5000 μM of ATP, 0-100 mM of KCl, 0-100 mM of NaHCO₃, 0-20 mM of EGTA, 0-1% of BSA, 0-50 mM of DTT (1,4-dithiothreitol), and 5-200 mM of buffer at pH6-8;
mixing the biological sample and the luminescence-base composition to produce a luminescence emission; and
determining an intensity of the luminescent emission to obtain an amount of urea in the biological sample, wherein the pretreatment of the biological sample is not required.

2. The method for measuring urea as claimed in claim 1, wherein the luminescence-based composition comprising a mixture of 0.1-100 U/mL of urea amidolyase (URL), 1×10⁻⁶-5×10⁻² mg/mL of firefly luciferase, 0.1-5000 μM of luciferin, 1 μM-20 mM of MgSO₄, 0.5-5000 μM of ATP, 0-100 mM of KCl, 0-100 mM of NaHCO₃, 0-20 mM of EGTA, 0-1% of BSA, 0-50 mM of DTT (1,4-dithiothreitol), and 5-200 mM of buffer at pH6-8.

3. The method for measuring urea as claimed in claim 1, wherein the luminescence-based composition comprising a mixture of 2-12 U/mL of urea amidolyase (URL), 1.4×10⁻³-2.8×10⁻³ mg/mL of firefly luciferase, 2-4 mM of luciferin, 5 mM-10 mM of MgSO₄, 0.75-1.5 mM of ATP, 30-60 mM of KCl, 10-20 mM of NaHCO₃, 0-8 mM of EGTA, 0.1-0.5% of BSA, 0-20 mM of DTT (1,4-dithiothreitol), and 25-50 mM of buffer at pH6-8.

4. The method for measuring urea as claimed in claim 1, wherein the buffer is selected from a group consisting of Gly-gly buffer, HEPES, Tris, Bis-Tris, Bis-Tris propane, MOPS, PIPES, phosphate, and borate.

5. The method for measuring urea as claimed in claim 1, wherein the buffer is Gly-gly buffer at pH 7.5.

6. The method for measuring urea as claimed in claim 1, wherein the concentration of urea is about 2.5 mM to 50 mM.

7. The method for measuring urea as claimed in claim 1, wherein the concentration of urea is about 2.5 mM to 37.5 mM.

8. The method for measuring urea as claimed in claim 1, wherein the luminescence-based composition is lyophilized form.

9. The method for measuring urea as claimed in claim 1, wherein the biological sample is serum or urine.

10. The method for measuring urea as claimed in claim 1, wherein the determining time is less than 10 min.

11. The method for measuring urea as claimed in claim 1, wherein the determining time is about 1 min.

12. The method for measuring urea as claimed in claim 1, wherein the volume of the biological sample is less than 20 μl.

13. The method for measuring urea as claimed in claims 1, wherein the volume of the biological sample is about 10 μl.
